(19) United States
(12) Patent Application Publication
(10) Pub. No.: US 2006/0255061 A1
(43) Pub. Date: Nov. 16, 2006

(54) Concentrated cleaner/disinfection system for biomatter contaminated materials

(75) Inventors: Marcia A. Frieze, Alpine, NJ (US); Allan S. Frieze, Alpine, NJ (US); Ron Amster, New York, NY (US)

Correspondence Address: Irving M. Fishman, North Tower, Suite 1422, 89 Headquarters Plaza, Morristown, NJ, 07960, US

(73) Assignee: Case Medical Inc.

(21) Appl. No.: 11/130,465

(22) Filed: May 16, 2005

(51) Int. Cl. B67D 5/08

(52) U.S. Cl. 222/64

(57) ABSTRACT

A system utilizing a concentrated cleaner and/or disinfectant and optionally concentrated lubricant or combinations thereof or concentrated multifunctional products within these classes; packaged in container sizes that are generally not greater than about 5 gallons each so as to permit greater flexibility in storage locations, especially at the area of use. In certain embodiments, the system further comprises an external empty bottle detection and alarm system. In certain embodiment, the system will also include a flow failure detection unit. In most preferred embodiments, both the “empty bottle” sensor and the “flow failure” are used, and preferably each has an independent ability to “lock up” the entire system to prevent further use of the system until the problem is corrected. In other embodiments a sediment avoidance feature is utilized, which is embodied either in the bottle design, in angling of mounting the bottle in a mounting bracket, or both.

### CROSS-REFERENCE TO RELATED APPLICATIONS

Not applicable.

### STATEMENT REGARDING FEDERALLY SPONSORED RESEARCH OR DEVELOPMENT

Not Applicable

### FIELD OF THE INVENTION

The present invention relates to concentrated cleaning systems, especially for cleaning instruments, devices, etc. having biomatter contamination. The invention also relates to concentrated cleaners for use in such systems, dispensing systems for use with such concentrated cleaners, and failsafe systems to avoid cleaning system failure. The invention particularly relates to the use of such systems for the cleaning of medical and veterinary instruments, industrial food processing equipment, and cleaning of waste lines having biomaterials transported therethrough from medical and non-medical facilities. The invention is most particularly related to systems, especially automated systems, for cleaning and disinfecting medical instrumentation using concentrated cleaning compositions.

### BACKGROUND OF THE INVENTION

In hospital and non-hospital medical institutions, various instruments and devices become contaminated with biomatter and medical waste and therefore need to be cleaned and disinfected before re-use. In instances where such instrumentation is relatively cheaply manufactured, one answer to this has been to develop single use disposable items. However, this is an unsatisfactory answer in that there are many such instruments and devices that are simply too expensive to merely be single use disposable items. In addition, in an increasingly “disposable” world, the available disposal sites are becoming fewer and fewer. Still further, even where disposal site availability would generally not be problematic, the disposal of such instruments and devices with potentially infections biomatter attached thereto presents an unacceptable environmental hazard. Thus, whether or not such instruments and devices are slated for re-use or are disposable, cleaning and disinfecting the same, before re-use or disposal is desired.

As a measure to reduce the volume of disposed materials and especially of biomaterial contaminated material (having very stringent disposal requirements), efforts have been evolving to develop systems and compositions for cleaning and disinfecting biomatter contaminated instruments, devices etc., so that expensive items may be re-used and/or inexpensive items may either be re-used or disposed of under conditions that are not as stringent as those for potentially hazardous or infections medical waste.

Typical use of cleaning and disinfecting systems in hospital contexts is to utilize various cleaning compositions, including without limitation, enzymatic detergents and non-enzymatic detergents, in manual, semi-automatic, and automatic operations, in various sequences, to clean (and possibly disinfect) the items in question before introducing the items into an autoclave or other final disinfection equipment. In current commercial hospital type settings, the storage container of the cleaner and/or disinfecting composition is generally a drum of in excess of 15 gallons, and pumped through an extensive amount of tubing or piping to the area of use. The use of such large storage containers generally means that the cleaning solutions are stored in storage rooms that are in other parts of the facility than the area of use. Having the storage container in another part of the facility means that an extensive amount of tubing or piping is needed in order to deliver the cleaning and/or disinfecting solution to the area of use. The inventor has personally seen situations where in excess of 100 feet of tubing is used in order to deliver the cleaning solution to the area of use. Such long delivery lines leave the system as a whole open to a number of potential failures that would not be typically detected, and would require extensive effort to correct, once detected. For example, long delivery lines mean that pump pressure may not be sufficient to deliver appropriate amounts of cleaner or disinfectant in a reliable manner resulting in insufficient cleaning. Long delivery lines can be problematic for replacement when the line is defective or clogged. Placing the storage container in a room other than the area of use means that it is difficult or overly time consuming for the storage container to be changed when it is or near empty. These are especially problematic when the cleaning and disinfecting system is an automated one where there is not a lot of attendant interaction with the system. One manner of dealing with the potential for empty or near empty storage containers is to periodically refill them from stock before they would typically be empty. While this can avoid a “dry” container problem, it means that solutions, many of which may be irritants and allergenic, will be exposed to the attendant responsible for refilling the storage container as well as introduces the possibility of spillage. Also, in some current commercial systems, the large storage containers have an internal fluid level sensor, typically a float valve that may be either visually seen or is connected via electronics to a display. The float valve or sensor is typically connected to a dip tube (used for withdrawal of the solution from the container for delivery to the area of use). When the container is empty or near empty, the container is refilled, or the dip tube is transferred from one container to the next. This again requires the attendant to be exposed to potentially irritating and potentially allergenic materials. In all cases where the solutions contain enzymes, the enzymes employed are ones designed to digest biological materials and therefore any direct contact with the attendant's skin would be unacceptable.

Thus, there is a substantial unmet need in having a cleaning and/or disinfecting system for use with biomatter contaminated instruments, devices, etc. that are free from the above deficiencies.

### OBJECTS OF THE INVENTION

It is therefore an object of the invention to provide concentrated formulations of cleaning and/or disinfecting and/or lubricant compositions.

It is another object of the invention to provide concentrated cleaning and/or disinfecting and/or lubricant compositions in package sizes that are sufficiently small to permit storage thereof at or near the area of use.

Yet another object of the invention is to provide such concentrated package units in sizes that may be mounted on or adjacent to the equipment that would be utilizing such solution.

Still another object of the invention is to provide a sensor of an “empty” or “near empty” bottle condition for use in conjunction with the concentrated solutions, particularly a sensor that is external to the container in question.

A further object of the invention is to provide an alarm system to notify the user or other attendant of the “empty” or “near empty” bottle condition.

An even further object of the invention is to provide a system that improves avoidance of sediment or particulate matter that may be present or develop in any of the solutions.

Another object of the invention is to provide a system in which the tubing or piping length between the storage container and the area of use is substantially shortened relative to current typical commercial usage.

An even further object of the invention is to provide a compact mobile cleaning and/or disinfecting unit that can be brought in complete into an operating room or other area on an as needed basis.

Yet still another object of the invention is to provide a waste disposal system for biomatter contaminated materials.

An even further object of the invention is to provide a mobile cleaning and/disinfection unit that can be readily brought to the a contaminated site for cleaning and disinfection of biomatter contaminated materials on site, without the need to transport such contaminated materials to a fixed decontamination facility.

Another object of the invention is to provide a means for readily cleaning and disinfecting waste lines through which biomatter contaminated materials passes.

Still another object of the invention is to provide a cleaning and/or disinfecting system for use in medical, veterinary, and laboratory settings, as well as in animal part processing facilities,

Still further objects of the present invention will be appreciated by those of ordinary skill in the art.

### BRIEF SUMMARY OF THE INVENTION

These and other objects of the invention are surprisingly achieved by a system utilizing a concentrated cleaner and/or concentrated disinfectant and optionally concentrated lubricant or combinations thereof or concentrated multifunctional products within these classes; each optionally with other suitable ingredients, packaged in container sizes that are generally not greater than about 5 gallons each so as to permit greater flexibility in storage locations, especially within short distances of the area of use or preferably at the area of use. In certain embodiments, the system further comprises an alarm system, most preferably external to the containers for detecting an “empty” or “near empty” bottle condition, or both, and advising the user or an attendant of such condition. In certain embodiments, the system will also include a flow failure detection unit, preferably external to the connecting tubing, preferably at or near the area of use end of the tubing, for detecting and alerting the user or attendant of a delivery failure. In most preferred embodiments, both the “empty bottle” sensor and the “flow failure” are used, and preferably each has an independent ability to “lock up” the entire system to prevent further use of the system until the problem is corrected. In other embodiments, a sediment avoidance feature is utilized, which is embodied either in the bottle design, in angling of mounting the bottle in a mounting bracket, or both.

### BRIEF DESCRIPTION OF THE DRAWINGS

FIG. 1 is a picture of a typical current system storage room in a hospital setting utilizing current large drum containers of cleaners/disinfectants.

FIG. 2 is a picture of a closeup of a container in use in a portion of the storage room of FIG. 1 that is not seen in FIG. 1.

FIG. 3 is a picture of a stationary mounting frame for a single container mount in current use.

FIG. 4 is a picture of another stationary mounting frame for dual container mounting in current use.

FIG. 5 is a picture of a single mount embodiment of the invention with a spring loaded weight sensor in the mounting shelf on which the shown bottle rests.

FIG. 6 is a picture of current drums in use with weighted probes.

FIG. 7 shows a cross section of an embodiment of the invention corresponding substantially to FIG. 5.

FIG. 8 shows the spring activated weight sensor of FIG. 7 without the bottle present with the sensor in a “not-empty” position.

FIG. 9 corresponds to FIG. 8 except that the spring activated sensor is in an “empty” or “near empty” position.

FIG. 10 is an orthogonal view of the shelf bracket of FIGS. 7 and 8 with the spring activated weight sensor in a “not empty” position.

FIG. 11 shows alternative bottle designs for use in the present invention that help assure that sediment or particulate matter is avoided in the solution that is withdrawn from the bottle while minimizing wastage.

FIG. 12 is a block diagram of a preferred embodiment having each of an empty bottle sensor, a flow sensor, and a failsafe shutdown switch.

### DETAILED DESCRIPTION OF THE INVENTION

The present invention is directed to a system for the cleaning and disinfection of instruments and devices that are contaminated with biomatter. For purposes of the present application:

biomatter shall mean (unless the context clearly requires otherwise) biological material resulting from medical procedures, veterinary procedures, laboratory procedures, and animal processing procedures;

medical procedures shall include, without limitation, those procedures performed in any medical setting, whether, without limitation, in a hospital, hospice, long term care facility, clinic, practitioner's office, ambulatory care facilities, surgery centers, or as an outpatient etc, whether or not performed by a licensed practitioner, a technician, the patient, or a caregiver;

practitioner shall include, without limitation, doctors, dentists, acupuncturists, and any other party who performs a procedure of any kind that results in an instrument, device or other material that becomes contaminated with biological material such that cleaning and disinfection thereof before reuse thereof (where applicable) or before disposal thereof as non-hazardous waste can be carried out;

veterinary procedures and practitioners shall mean the same as medical procedures and practitioners, except that the patient is an animal;

laboratory procedures shall mean any procure conducted by a laboratory for diagnostic or analytic purposes (inclusive of research laboratories) that results in material that becomes contaminated with biological material such that cleaning and disinfection thereof before reuse thereof (where applicable) or before disposal thereof as non-hazardous waste can be carried out; and

animal processing procedures shall mean any procedure performed on any animal or animal part, other than those within veterinary procedures, and shall specifically include processing animals or animal parts for food, clothing, etc., such that the procedure results in material that becomes contaminated with biological material such that cleaning and disinfection thereof before reuse thereof (where applicable) or before disposal thereof as non-hazardous waste can be carried out.

For convenience purposes, the invention will be described in the context of a hospital or large medical facility setting. However, the teachings and suggestions of the specification are equally applicable to the other settings mentioned herein (including without limitation, hospitals, hospices, long term care facilities, clinics, ambulatory care facilities, surgery centers, dialysis centers, a practitioner's office, a patient's residence, the corresponding locations involving the care and treatment of animals (including shelters), laboratories and facilities performing animal processing procedures. It is also applicable to retail and wholesale fish and meat shops if desired.

The present invention is system for use of a generally small storage container, generally no greater than 5 gallons, more preferably no larger than about 4 gallons, more preferably no larger than about 3 gallons, still more preferably no larger than about 3 gallons, most preferably no larger than about 2.5 gallons each, although smaller containers such as 1 gallon containers are perfectly suitable. The system can use just a cleaner as a pre-soak or as a pre-wash before introducing the material to be cleaned into a chemical or heat (or both) disinfecting procedure. In either case, the cleaning solution may have an enzymatic component therein or the enzymatic component may constitute a separate solution, or no enzymatic component need be used at all. In the case where the material to be cleaned has moving parts that might be subject to corrosion from the cleaning and disinfecting procedures, a lubricant component is also used, which depending upon the particulars of the materials involved may be included in the cleaning or disinfecting solution, but is generally preferably a separate solution. All manner of such solutions are well known in the art and may be employed in the present invention. However, especially useful in the present invention are substantially concentrated solutions.

Thus, in a preferred embodiment, the cleaning and disinfecting system of the invention has three separate solutions, a cleaning solution, a disinfecting solution, and a lubricant solution. As stated, each of these solutions may be formulated to have more than one of these functions, or one or more may be omitted without departing from the spirit of the invention. Especially preferred are solutions that in use are recommended for dilution at in the range not more than 1 oz/gallon (although less concentrated solutions may be used), preferably in the range of 0.05 oz/gallon to about 0.75 oz/gallon, more preferably about 0.1 oz/gallon to about 0.5 oz/gallon, and may be used at dilution amounts of 0.125, 0.2, 0.25, 0.33, 0.4 oz/gallon. Typical such solutions include SUPERNOVA MULTIENZYMATIC Cleaner (available from Case Medical Inc., Ridgefield, N.J.) and PentaZyme (available from Case Medical Inc., Ridgefield, N.J.). Other cleaner and disinfecting solutions from other suppliers will be known to those of ordinary skill.

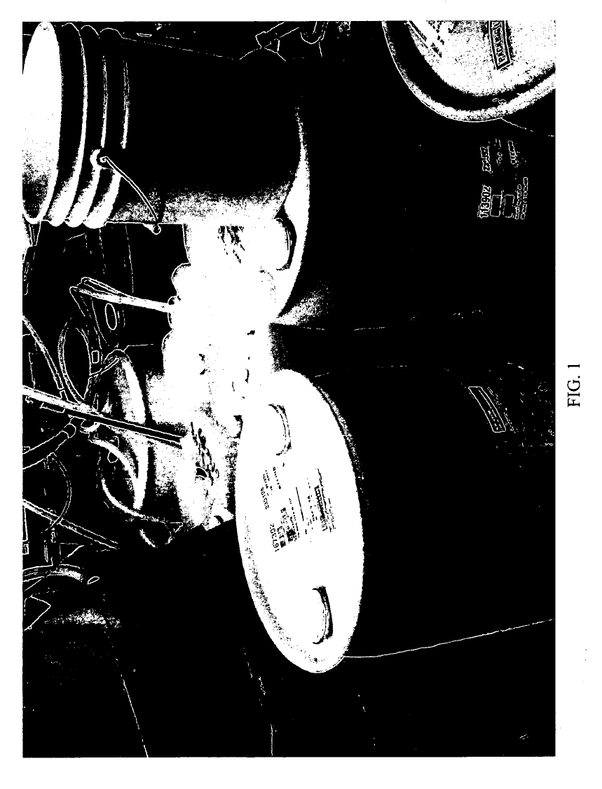

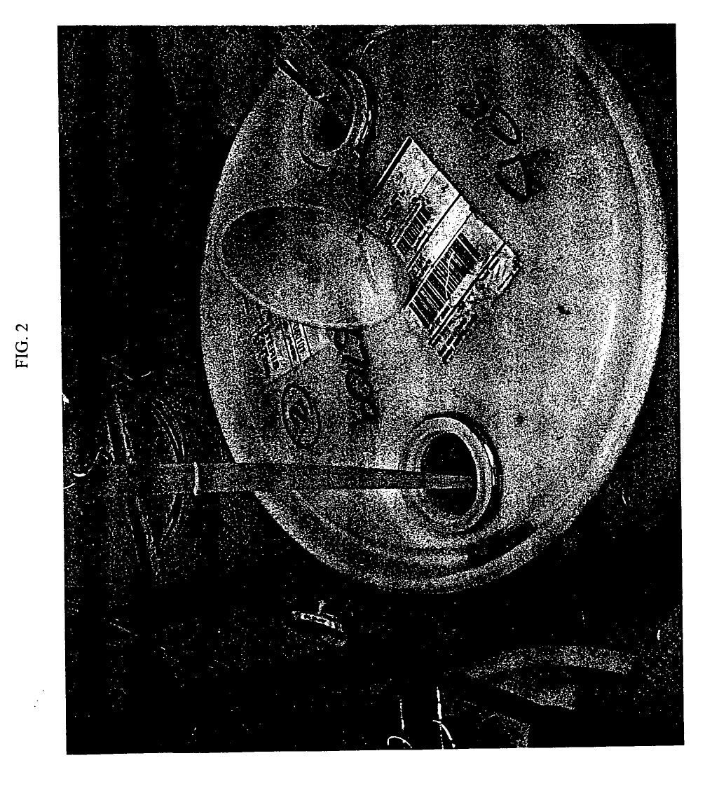

As discussed above, the existing commercial setups are rather bulky and cumbersome and require a separate storage room for the reservoir containers. FIG. 1 is a picture of an actual current system in use in a large metropolitan hospital, while FIG. 2 is a closeup of one of the containers in that room (not shown in FIG. 1). As can be readily seen, the current systems utilize very large containers that are not conducive to being present at or near the area of use, utilize large amounts of tubing to deliver the solutions to the area of use, and are unsealed when being used. As discussed above, these conditions are not desirable and improvements in the system have long gone unmet.

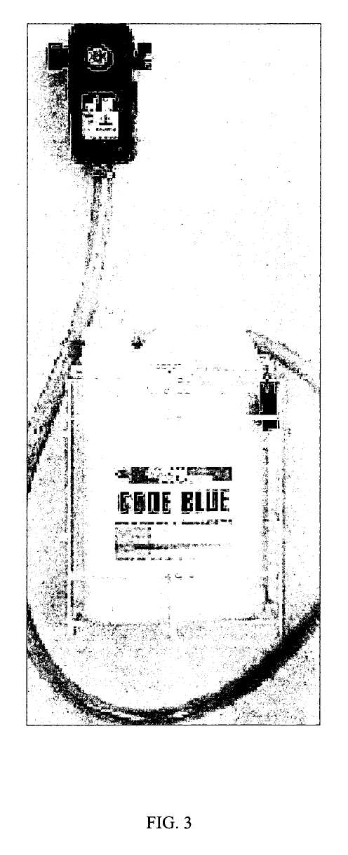

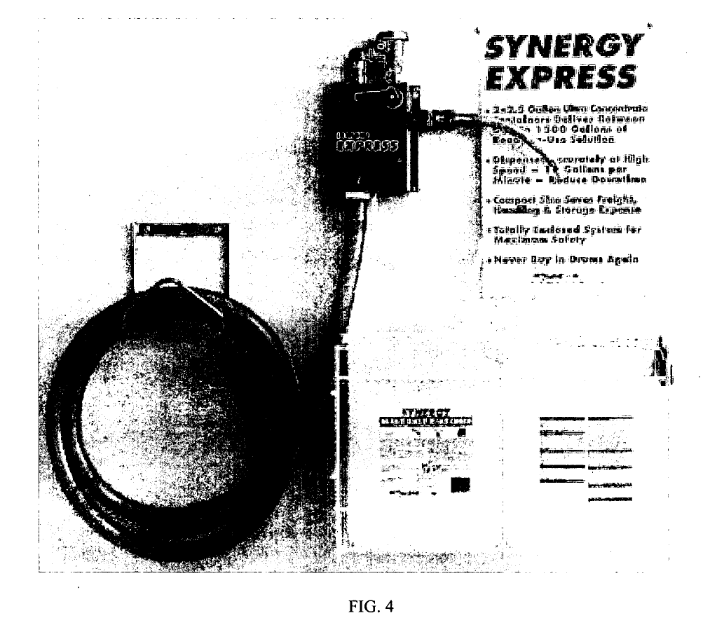

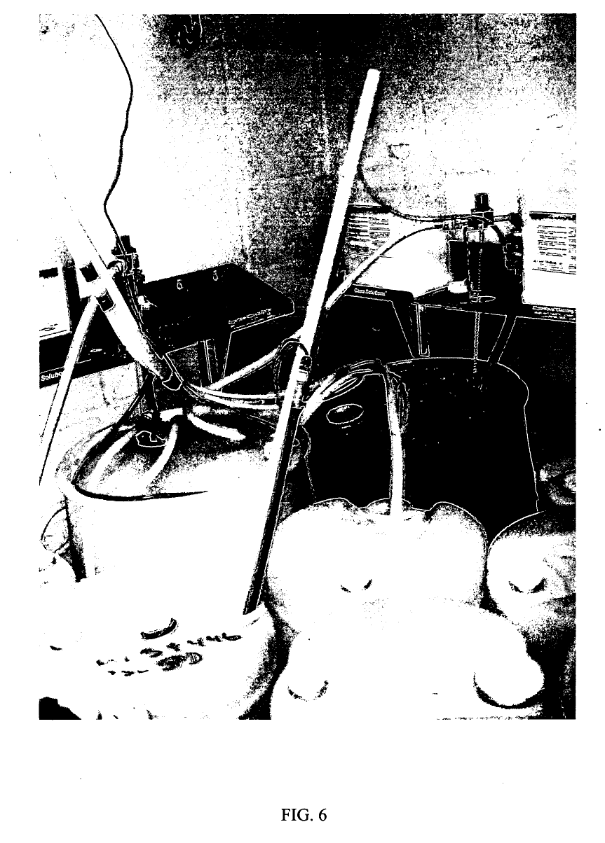

FIG. 3 shows a current small container bottle mounted within a fixed metal cage in a storage facility, while FIG. 4 shows a dual container system of the same type. Neither of these have external sensors for detecting an “empty” or “near empty” bottle condition. Any indicator of an empty bottle condition in these older systems is either one that is internal to the container, sensors that are environmentally unfriendly, sensors that are expensive and require opening the containers to remove the sensors for reuse, or simply rely on the visual appearance of the container to an attendant. The present invention utilizes such small sized containers in single or multiple arrangements in combination with external sensors to determine whether the bottles are at or near an empty condition. Because the sensors in the invention method and system are external, the containers need not be opened when replacing empty bottles making the replacement easier, more reliable, and safer to the attendant who has to replace the empty bottle. In one embodiment of the invention, when dual bottle systems are used and both containers have the same solution and each is simultaneously connected to a manifold, which manifold receives a signal from an “empty bottle” sensor, the manifold automatically switches in response to that signal between the bottles as the source of the solution to be delivered. In an alternate embodiment the two solution bottles contain different solutions, with each being connected to its own pump and delivery tube, which alternate embodiment is more desirable in situations where user or attendant attention is more closely involved, mobility of the system is desired, or the system will be used in only small portions of the day, so that assuring a filled source container is present is more likely to be attended to at the beginning of a day and need not be done again for that day. Sensors internal to the bottles can be utilized, but preferably external sensors are more desirable as discussed. FIG. 6 shows weighted probes in use with large drums to determine the container content level.

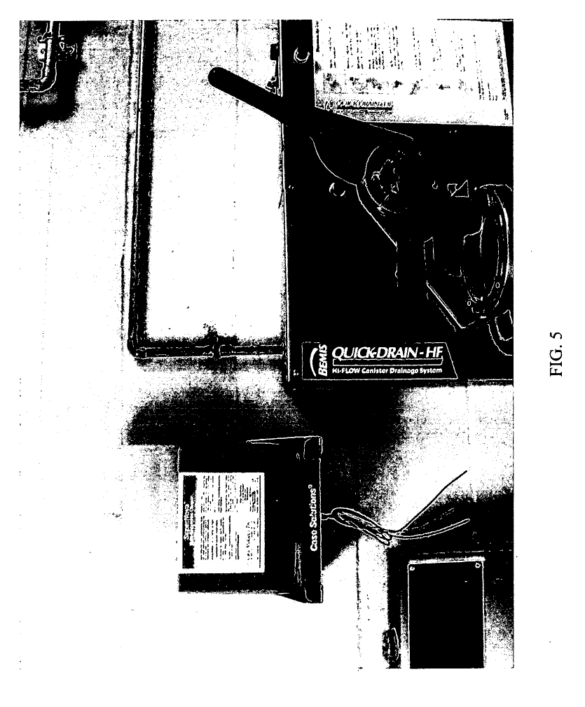

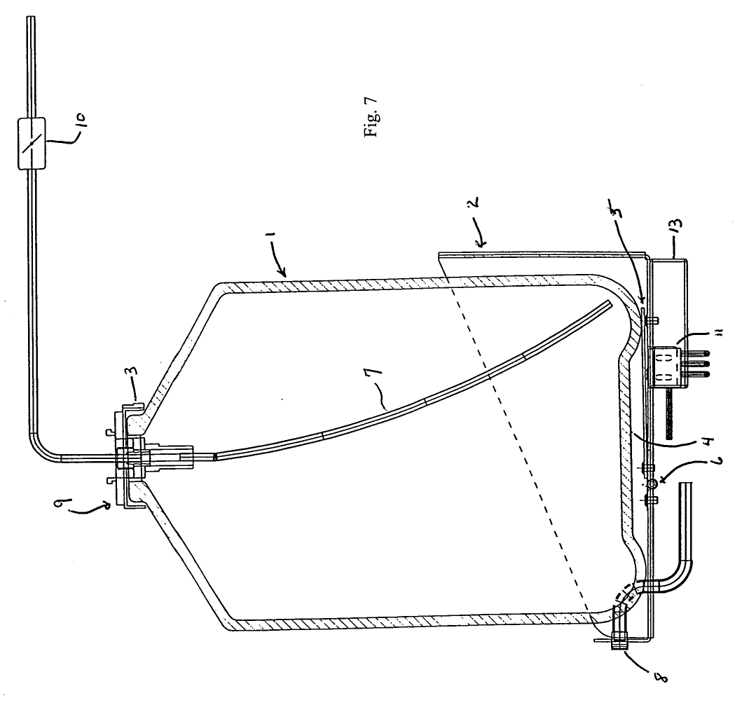

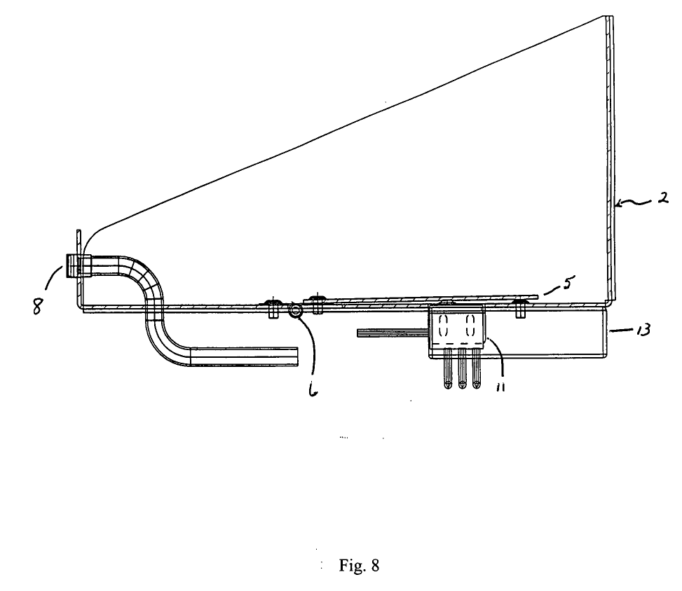

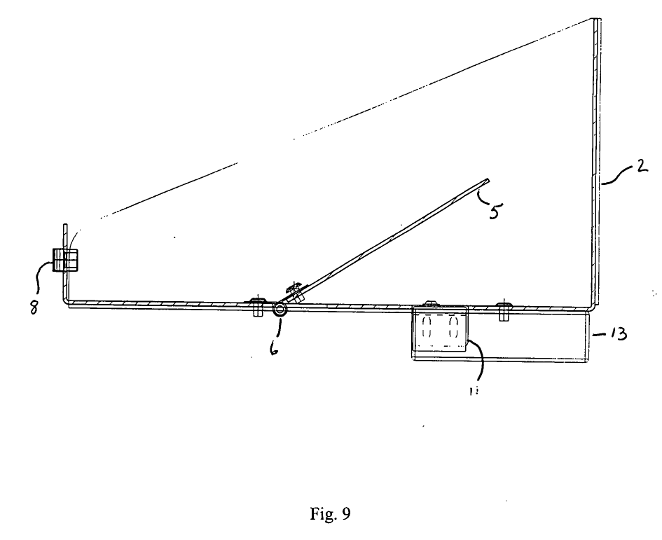

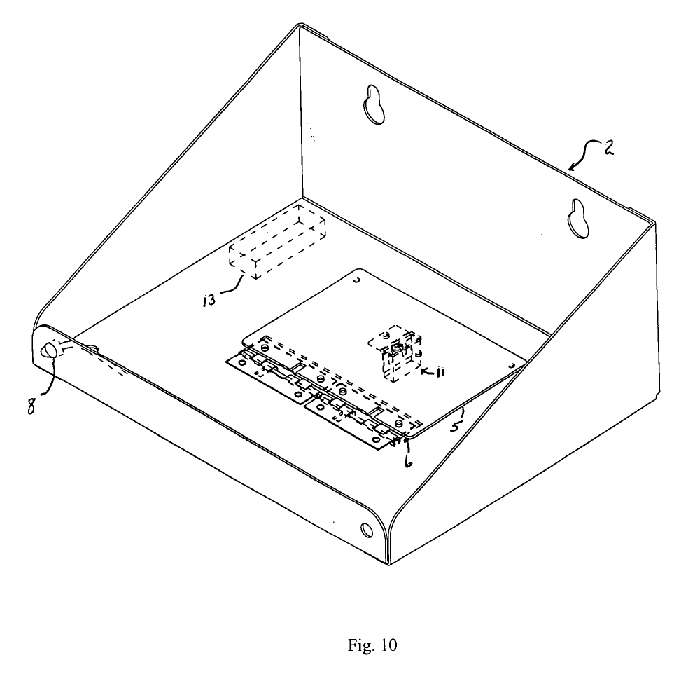

FIG. 5 shows a partially filled container of the invention resting on a wall mounted shelf having, as the external sensor of the present invention, a spring loaded weight sensor. While a weight sensor of this type is one preferred embodiment of the present invention, any other “empty” container condition or “near empty” container condition sensor is perfectly acceptable, and such sensors means will be readily apparent to those of ordinary skill. Such other sensor means include a plate supported by a partially filled flexible bladder to bias the plate to an empty position but which is displaced under the weight of a “not-empty” container to a “not-empty” position. In these cases the “not-empty” position allows for either the contact with or disconnect from a switch such that when the external sensor is displaced to the empty position, a signal is generated to alert the user or attendant. Another alternative sensor device for use in the present invention includes a piezoelectric material which changes its electrical conductivity depending upon the pressure exerted against a surface thereof. In such a case, a portion of the shelf or other surface upon which the container rests is made of such piezoelectric materials and is connected with suitable standard circuitry known in the art to generate a suitable “empty bottle” condition. Yet another alternative for an empty bottle condition is to fix the volume of dispensed solution per use and knowing the volume of the container contents have the number of uses counted until a set number is reached and then upon reaching such a number of uses alerting the user or attendant of the need to replace the empty container. An even further “empty” condition sensor is an optical one which shines a light through a lower portion of the container and solution and thus defines a “not-empty” transmittance. When the contents drops to a point where all or part of the light beam no longer passes through the solution, the difference in transmittance is interpreted as an “empty” container condition and a signal is generated to alert the user or attendant of such condition. Still other variations will be appreciated by those of ordinary skill. The preferred sensor (a spring loaded weight sensor) is shown more specifically in FIGS. 7-10. FIG. 7 is cross-section of the sensor shelf shown in FIG. 5. In FIG. 7, container 1 rests within shelf bracket 2. Shelf bracket 2 may be mounted on a vertical or horizontal surface, which may be a wall adjacent or near the area of use, or may be a portion of the unit to which the contents of the container are ultimately dispensed, or may be a mobile cart. In the embodiment shown in FIGS. 7-10, the container is in the upright position with the cap 3 at the top of the container. In this embodiment, the container bottom 4 rests in whole or in part against a spring-loaded weight sensor plate 5 movably attached to the shelf bracket 2 in a hinged relationship therewith, generally biased to an “empty condition” position by spring 6. When the weight of the container and contents is above a specified weight, the weight forces the spring-loaded weight sensor to a “non-empty” position. As the contents of the container is dispensed (through dip tube 7) the weight of the container and remaining contents thereof reach a point where the spring-loaded sensor can no longer be held in the “non-empty” position, and the spring-loaded sensor moves to an “empty position”. FIG. 8 shows the shelf bracket with the spring loaded weight sensor without the container 1, while FIG. 9 shows the same embodiment without the container 1, but the spring loaded weight sensor in an “empty” or “near empty” container position. Sensor switch 11 is connected to a power source, which may be an outlet for current, or a battery or both. In preferred embodiments a battery 12 (not shown) is used and held on-board in battery holder 13, which may be located in any desirable location. In highly preferred embodiments, the system will have a “low battery” detector and on reading such a condition will activate an audible or visible signal. Such “low battery” detection means are well known in the art and any such system or device will be suitable for use in the present invention.

Other, more complex external sensors of an “empty container” condition are perfectly suitable for use in place of the spring loaded weight sensor. These include, without limitation, weight sensors, such as scales of various kinds; electromagnetic sensors, such as those measuring a difference in visible, ultra-violet or other electromagnetic transmittance or absorbance when passing through a specified portion of the container and its contents to a detector; etc. While internal sensors in the containers may also be utilized for the invention, they are less preferable in that they are more expensive to produce, and have their own disposal issues if not re-used and if re-used require removal from the containers for re-use. Thus, in preferred embodiments of the invention, sensors external to the containers are used for detecting an “empty container” condition.

Spring loaded weight sensors for use in the invention can be those which displace the container relative to the shelf or bracket upon which the container rests when in use (as shown in FIGS. 7-10) or can be one where the shelf or bracket upon which the container rests is displaced in the vertical direction along with the container relative to a bracket in which (or wall on which) the shelf is mounted. Typical weight displaceable movable carriages are frequently found in use with barbeque grills using propane tanks.

In a highly preferred embodiment, the sensor of an “empty container” condition is connected to an alarm system to alert the user and/or attendant that the container in question is empty or at a nearly empty condition. Most advantageously, a dual sensor and alarm system is utilized to detect and alert both a “near empty container” and “empty container” condition. The alarm may be visual or audible, and advantageously is indicated at both the container and at least one of control panel for use of the system and the area of use which may be in a more advantageously located position. Any visual or audible alarm system can be used, but is most preferably at least a digital display. FIGS. 7-8 illustrate this portion of the invention by use of an LED display or buzzer 8.

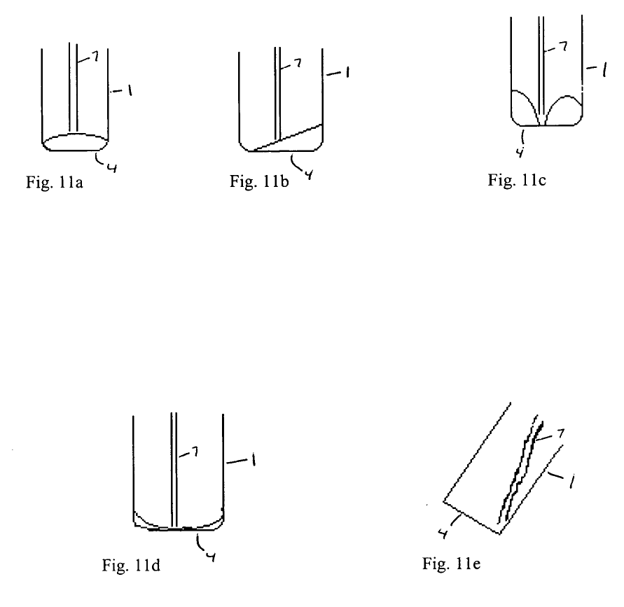

Most advantageously, in preferred embodiments, the invention system also contains a cutoff switch to which the “empty container” sensor is connected, either physically or electronically. Where single containers are used, upon detection of an “empty container” signal, the cutoff switch locks up the system so that it cannot be used until the “empty container” condition is remedied. In systems where multiple containers of the same solution are simultaneously connected, the cutoff switch will automatically switch to a second container of that solution that does not have an “empty container” condition detected, and will only lock up the system if all of the containers of the same type are being detected as “empty”.

Where desired the “empty container” condition can be set so that the container is truly “empty”, or to assure lack of system failure, the “empty container” condition detection can be set so that there is actually a small amount of the container contents still remaining when the condition is detected. The latter is especially advantageous when there is some concern that there might be sediment in a solution being used. Having a small amount of the solution still found in the container when the container is to be changed allows one to place the dip tube for withdrawing solution at a finite distance above the very bottom of the container when in use, thus allowing any sediment to settle out of the solution without being taken up by the dip tube. Advantageously, but not required, the containers have lower internal portions (bottoms) that are not completely horizontal and flat (when the container is in its normal storage position of use). FIG. 11 shows a number of types of container cross-sections that are designed to reduce or eliminate the sediment that may be picked up by the dip tube without creating an overly large container-contents wastage. In FIG. 11a, the dip tube rests against an internally raised convex surface whereby any sediment present will typically fall to the perimeter valley between the raised potion and the container wall. In FIG. 11b, the container interior has a raised angled bottom (which may be a single portion or multiple portions and which may be flat, concave, or convex), with the dip tube resting on a portion of the raised area at a specified distance from the bottom of the container interior. FIGS. 11c-11d illustrate additional non-limiting arrangements. In addition, the same sediment avoidance can be achieved with a flat bottom container where the dip tube rests on the bottom, by having the container be used in a tipped condition so that relative to a level resting position, the dip tube is raised slightly above the lowest point of the container interior when tipped, as shown in FIG. 11e. Other arrangements will be readily apparent to those of ordinary skill in the art.

Another advantageous portion of the invention is the use of a barbed cap or Lauer Lock type of closure 9 on the container. When used, the barbs may be located on either side of the cap, i.e. being internal or external of the container or both when the barbed cap is in place on the container. This is made possible and cost effective in that the “empty container” sensor is external to the container so that it need not be removed for reuse or disposal purposes. A commercially available closure of this type is DuraSeal™ (available from DEMA Engineering Company, St. Louis, Mo.), shown in FIG. 7. Other suitable container closure systems will be apparent to those of ordinary skill in the art. The use of these types of closures means that the container need never be opened in a manner that would subject the party opening the container to contact with the contents of the container.

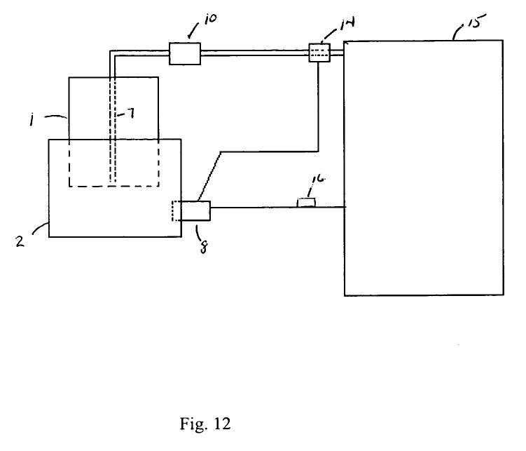

In highly preferred embodiments, in addition to an empty container sensor (located near or at the container), an additional flow sensor 14 (see FIG. 12) is employed at some point before the tubing connects the container to the area of use 15, preferably just prior to entry to the area of use. This is particularly preferred where the area of use is an automated washing and/or disinfection unit. The advantage achieved by this additional sensor is to detect and shutdown the system when there is a solution flow problem detected even though the container source of the solution is not empty. A pressure sensitive flow detector (such as for example a sphygmomanometer) can be readily used. Other external fluid flow detection means will be apparent to those of ordinary skill in the art, and include, without limitation, those utilizing optical, electrical, and/or ultrasonic detection means. When such fluid flow detectors are used in addition to the “empty container” detectors, and a failure of either condition is detected, a preferred embodiment sets off an alarm 8, with a visual or audible or electronic signal or combination thereof to alert the user or attendant and in highly preferred embodiments, simultaneously locks the system via lockout mechanism 16 until such fault condition is corrected. In more complex systems of the invention, when redundant multiple containers and connecting tubing is used, the invention system in detecting a fault in either condition sets off the alerting alarms and attempts to switch to the backup not faulting setup. Where all backup set ups are also showing failure signals, the system is then shut down until the faults in at least one of the redundant set-ups are cleared. FIG. 12 shows a block diagram of a single container system having both an “empty container” sensor and a fluid flow sensor. In further highly preferred systems, a check valve 10 (see FIG. 7) is placed at some point in the fluid flow path to prevent backflow into the container. As with the other safety features of the present invention, this check valve is preferred to be external to the container, but if desired, a check valve within the container is also suitable for the present invention.

The container need not be positioned in an upright dispensing position as shown in the various figures, but may be in an inverted position, using a gravity feed or a pump. In such inverted positions, dip tube 7 is generally not used, but if desired to avoid sediment, a small segment of dip tube may be used so that the sediment, if any will settle to areas that will not empty from the container. This type of arrangement is similar to the typical “office water cooler” dispensing, with the container neck having a seal which opens only when engaged with the dispenser. In these arrangements, the container neck or shoulder is disposed against the weight sensor holding in the “not-empty” position until sufficient container contents have been discharged so that the container and its contents are no longer sufficient to hold the sensor in place. An alternative container is one that is a flexible bag that collapses as the contents are dispensed. This is particularly advantageous in systems with the inverted container, and especially with chemical systems for which ambient air intake (to balance pressure changes due to dispensing of the container contents) is problematic.

The invention system described above is made especially possible by use of significantly concentrated solutions in the cleaning and disinfecting regimen of the implements to be so cleaned and/or disinfected. This avoids the concern of appropriate dissolution of solid blocks of cleaners, while at the same time eliminates the need for large storage facilities and cubersome container changing necessitated by the large 15 gallon and larger drums in general current use. While generally the same components are used in the present invention, they are typically present in concentrations which are 2 to 5 times as high, permitting the use of smaller amounts per treatment cycle, the use of smaller containers of the feed solutions, and thereby all of the advantages of the present invention. Nonetheless, currently used solutions that are not as concentrated as set forth in the present invention are perfectly capable of being used with the present invention system using the smaller (under 5 gallon) containers but this will require much more frequently changing of the containers because they will be emptied much more frequently.

When the present system is intended to have more than type of solution connected to it at a time, it is preferable to have the solutions, the respective hoses, and the connection points color coded so that connecting an inappropriate solution to a particular connection point or tube is avoided. Indeed, even when single solution systems are used, they are preferably color coded as more than one such single solution systems would likely be in use in any particular facility or cleansing and disinfection operation.

The systems as described above can be used in connection with automated equipment as well as dispensing the respective cleaning and disinfecting solutions to manual areas of use. It can also be discharged into waste lines to clean them by automatically discharging a charge of cleaner and/or disinfectant either at regular intervals or when the waste line is active. In one embodiment of this variation, the rushing of the waste line paste a nozzle connection point into the waste line creates suction in the nozzle, which then discharges the cleaning or disinfecting solution. In another embodiment, the flow inside the waste line is detected and at a designated time thereafter, a charge of cleaner and/or disinfectant is automatically dispensed into the waste line. Optionally, when enzymatic materials are used, the discharge of the enzymatic materials into the waste line activates a further discharge into the waste line (at an appropriate downstream point, at an appropriate subsequent amount of time) of a deactivator of the enzyme so as to eliminate any active enzyme from being discharged into the environment.

We claim:

1. A concentrated cleaner and/or disinfection system for biomatter contaminated materials comprising:
(a) a first container having a composition for at least one of cleaning, disinfecting, and lubricating a material contaminated with a biomatter contaminant;
(b) a first sensor means for detecting when said container is empty or near empty or both;
(c) delivery means from said container to an area of use of said composition in the cleaning, disinfecting, and/or lubrication of said material;
(d) optionally, an alarm to alert at least one of a system user or a system attendant of at least one of a container empty and container near-empty condition.

2. The system of claim 1 wherein said first sensor means is external to said container.

3. The system of claim 2 wherein said first sensor means is selected form the group consisting of a weight sensor, a counterweight, a weight sensitive piezoelectric sensor, an optical or infra-red sensor, a hydraulic weight sensor, or combinations thereof.

4. The system of claim 3 wherein said weight sensor is spring loaded.

5. The system of claim 4 further comprising a platform on which said container is placed for use in said system wherein said spring loaded weight sensor is contained in a portion of said platform below the position of the container when the container is placed on said platform or said platform being movably connected to a spring loaded member; such that under the weight of said container on said platform, said spring loaded member is displaced so that said weight sensor is in a first position and upon dispensing of said container contents, the weight of said container and contents is reduced sufficiently to allow said spring loaded member to be displaced to a second position; said second position indicating a substantially empty container condition; said first position indicating an other than a substantially empty container condition.

6. The system of claim 2 wherein said first sensor is an optical or ultraviolet light sensor.

7. The system of claim 1 further comprising an alarm system that alerts a user of the system or an attendant of the system that an empty container condition has been detected, said alarm system being at least one of an audible alarm and a visible alarm.

8. The system of claim 1 further comprising a second sensor that is capable of detecting fluid flow through said delivery means.

9. The system of claim 8 wherein said second sensor is located at or near said area of use.

10. The system of claim 8 wherein said second sensor is a pressure sensor located external to the delivery means and capable of detecting adequate fluid flow through said delivery means.

11. The system of claim 1 wherein said container has a barbed cap or Lauer lock mechanism to permit engagement of said delivery means to said container without exposing said container contents to the environment.

12. The system of claim 1 wherein said container has a dip tube therein for dispensing the fluid contents of said container, said dip tube having a first end at a dispensing end of said container and a second end at or near the bottom of the said container and said container has one or more concave, convex, or substantially flat angled bottom so that the residual fluid in said container when the second end of said dip tube is no longer submerged in said fluid, is reduced over that when a flat bottomed container is used.

13. The system of claim 1 wherein said container has a substantially flat bottom and has a dip tube therein for dispensing the fluid contents of said container, said dip tube having a first end at a dispensing end of said container and a second end at or near the bottom of the said container, where said container is, when in use, is placed in a tipped arrangement relative to the horizontal so that said dip tube can be placed further down into the container and the residual fluid in said container when the second end of said dip tube is no longer submerged in said fluid, is reduced over that when said container is not tipped.

14. The system of claim 1 further comprising at least a second container of the same solution as said first container; said empty container sensor upon detection of an empty container condition, redirects said delivery of said fluid from said first container to delivery of said fluid from said second container.

15. The system of claim 1 wherein said alarm system includes a system shut off signal when said first sensor indicates an empty container condition.

16. The system of claim 1 wherein said container is not greater than about 5 gallons.

17. The system of claim 1 further comprising a check valve located in the path of fluid flow such that said check valve prevents backflow of said fluid through said system.

18. A method of cleaning or disinfecting a biomatter contaminated material comprising dispensing a concentrated cleaning and/or disinfecting, and/or lubricating composition via the system of claim 1.

19. The method of claim 18 wherein said material is a medical instrument, a veterinary instrument, a laboratory instrument, a biowaste material, a discharge pipe, a commercial food processing facility implement, or a commercial food processing plant disposal pipe.
